(19) United States
(12) Patent Application Publication
(10) Pub. No.: US 2012/0277527 A1
(43) Pub. Date: Nov. 1, 2012

(54) Stereoscopic Imaging from a Conventional Endoscope with single lens camera

(76) Inventors: SANDEEP SOOD, TROY, MI (US); BEENA GAIND SOOD, TROY, MI (US)

(21) Appl. No.: 13/098,407

(22) Filed: Apr. 30, 2011

(51) Int. Cl. A61B 1/04

(52) U.S. Cl. 600/109

(57) ABSTRACT

The present invention discloses an LCD device and viewing eyewear for obtaining stereoscopic images with conventional single optic channel endoscope using a single lens camera. LCD device comprises two segments each of which is activated by a controller through a wired or wireless connection and is installed between the endoscope and camera without affecting the optic path length. Images are viewed on a standard monitor using eyewear comprising LCD shutters on the left and the right side which are synchronized to the segments in the LCD device by a controller. The LCD shutters on the eyewear are in the upper inner field of vision of each eye. This allows the user, usually a surgeon, to have stereoscopic view when looking at the monitor, but clear view when he has to look at the operative field. The disclosed eyewear has a sterile attachment that the surgeon can use for mounting, un-mounting or adjusting the eyewear.

### CROSS REFERENCE TO RELATED APPLICATIONS

Not Applicable

### STATEMENT REGARDING FEDERALLY SPONSORED RESEARCH OR DEVELOPMENT

Not Applicable

### DESCRIPTION OF ATTACHED APPENDIX

Not Applicable

### BACKGROUND OF INVENTION

This invention relates generally to the field of stereoscopic imaging using conventional single optic channel endoscopes with single lens camera and viewing eyewear for use in a surgical setting.

To develop stereoscopic perception, brain needs to have two perspectives of the object, one from the left and the other from right side. This may be done using two separate cameras that provide separate side by side images and a method of blanking out alternate images from a left and right camera so the viewer sees the alternate images with a left eye followed by a right eye. This can be achieved using endoscope with two optic channels each feeding into a separate camera. Alternatively, in the known systems today, a single optic channel endoscope may have a beam splitter at the exit pupil that feeds the left and right cameras to provide left and right images for viewing. Various combinations of additional prisms, mirrors, refracting lenses have been positioned either in the entry pupil or the exit pupil to achieve the same objective. In all these cases two focusing lenses are needed instead of one as there are two separate sets of image rays or image paths that create two separate images. These systems may instead have a single camera head with two chips to record the right and the left perspective. Takahashi in U.S. Pat. No. 5,522,789 described a stereo-endoscope with a single optical axis but a pair of imaging devices. Becker in U.S. Pat. No. 5,944,655 described a 3D video endoscope with two lenses at the front end, a single beam path alternately used by the beams by an optical switch projecting image onto a single camera. Schoolman 1984 U.S. Pat. No. 4,651,201 disclosed an endoscope with a beam splitter and two oculars for two camera heads. Chaleki et al 1996 U.S. Pat. No. 5,751,341 description of a 3D endoscope system also includes possibly a two camera system from the description of the text.

Such systems are plagued with three problems. First, the size of the endoscope having two optic channels is significantly larger and may be prohibitive to be introduced into a body cavity because of higher risk of injury. Second, the two cameras or a single camera head with two chips add to the weight of the endoscope making it difficult to perform delicate operations. Thirdly, it is difficult to precisely tune two cameras or the two chips in a single camera to same quality, color, hue and tint of the images. As a result slight difference in these properties of the images projected to the right and the left eye cause excessive eye strain and may be prohibitive in long operations.

Proposals have been made in the prior art for stereographic imaging using only one camera system and single optic channel endoscope. These systems achieve their objective by placing a shutter over half of the optic path between the object and the lens or between the lens and the camera chip and move the shutter from side to side.

All of these systems shift the principal ray from the center of the entry pupil to one side. Thus, different perspectives, i.e., a left perspective and a right perspective, are produced which can provide stereoscopic viewing. These systems have some draw backs that preclude their use in existing convention endoscope and single lens camera systems.

The concept of utilizing colored filters in the camera and a passive eyewear to generate stereopsis was disclosed in U.S. Pat. No. 3,712,199 to Songer 1973. Using complimentary color filters is associated with significant color loss in the image especially red color. Further the viewing glasses as described would constrain the view of the surgeon to be through the color filters when looking down at the patient or to the sides causing unnatural color.

Lia, 1993 U.S. Pat. No. 5,222,477 describe a system comprising an aperture plate interposed in the optical path of the camera and the adjacent lens assembly. The aperture plate has two pupils on each side of the optic axis and a shutter assembly to alternate exposure through each pupil. Since the total size of the pupils is smaller than the lens it has the drawback of reducing the image intensity.

In Shipp 1995 U.S. Pat. No. 5,471,237, the optic shutter system is placed between the objective of the endoscope and the output and is an integral part of the endoscope. It therefore cannot be used for a conventional existing endoscope.

Songer 1997 U.S. Pat. No. 5,606,363 describe a camera with a single lens with a dual aperture light valve as an integral part of the optics for generating 3D images. Such a system cannot be used on existing endoscope systems and cameras.

Watt 1999 U.S. Pat. No. 5,914,810 disclose use of more than two optical shutter elements distributed from left to right in the shutter device to control the light transmission. Presence of more than two optical shutter elements adds to the complexity to the system. The space between the shutter elements will reduce the total amount of light transmitted and intensity of the image.

Michalca 1999 U.S. Pat. No. 5,964,696 have an optical shutter with two apertures spaced apart disposed between the endoscope and the camera in a separate detachable housing. This system has the same drawback of reduced image intensity since the total area of the two apertures is less than that of the lens. In addition the housing adds optical path length and therefore may not be compatible with use in existing endoscopes.

Greening 2000 in U.S. Pat. No. 6,151,164, describe a system wherein an opaque leaf moves from side to side. The attachment with mechanics to drive the opaque leaf will require modification to the endoscope optics since additional optical path length would be required when the attachment is placed between the endoscope and the camera.

Huang 2003 U.S. Pat. No. 6,580,557 presented an invention of a single lens instantaneous 3D image taking. The aperture disc has three off axis hole generating three different image perspectives on the camera. Again like Lia's patent the size of the apertures is considerably less than that of the lens. The major drawback is reduced image intensity.

Weissman 2003 U.S. Pat. No. 6,664,935 describe a method for centering the image blocking element to obtain improved stereopsis. The need for a housing between the camera and the endoscope, to move the image blocking element, will change the optic path length from the endoscope to the camera and therefore will not be compatibility for use with existing scopes. Use of plurality of segment in a liquid crystal image blocking element, in an alternative embodiment, has the drawback of reducing the image intensity.

Costales 2004 U.S. Pat. No. 6,683,716 show a method for obtaining 3D imaging using a single lens that involves multiple active and passive polarizing filters to separate the right and left images in a microscope. Multiplicity requires that these are build into the endoscope or its camera. This would preclude using these in an existing system such as our invention can be. Further, the use of half wave retarder in one half of the optic path is going to change the path length in that half affecting the sharpness of the image from that half of the optics.

Gim 2004 U.S. Pat. No. 7,068,416 disclosure of a three dimensional imagery where variable focal length is used to derive information about the object. This type of process is not likely to be possible in existing endoscopes.

Cho et al 2010 U.S. Pat. No. 7,751,694 variable focus microarray system to generate 3D endoscopic imaging cannot be used for existing endoscopes.

In brief, each of the prior art technology suffers from one or more of the following deficiencies. 1) Optical shutter with two spaced apart pupils, or multiple segments in Liquid Crystal shutter cause loss in image intensity. 2) Use of an additional housing or adapter, between the endoscope and the camera to control the mechanical elements of the shutter or for aligning the shutter change the path length between the endoscope and the camera requiring modification in the optics to maintain the same image size and magnification. This precludes use for conventional existing endoscopes with a single lens camera. It further adds weight to the entire system and is uncomfortable to use for the surgeon. 3) The right and left side of the optical shutter have different path lengths. This would cause a difference in the sharpness of the image from the two sides. 4) Optical shutter is an integral part of the optics of the endoscope or the camera, precluding use for conventional existing endoscopes with a single lens camera.

### BRIEF SUMMARY OF INVENTION

The present invention avoids the necessity of changing the optics of the endoscope or the camera, since it does not change the path length, as there is no additional housing or adapter between the endoscope and the camera. It can therefore be used with existing endoscopes and needs only a single image path between an image and a camera.

Object of the invention is to obtain stereoscopic imaging with a conventional single optic channel endoscope using a single lens camera. The disclosed optic shutter comprises a single Liquid Crystal Display (LCD) device with only two segments. The left and the right segment of the aperture can be individually activated by electrical impulse to shut off light transmission through the respective segment. The LCD device is placed between the endoscope and the camera without the need for a separate housing. The left image perspective is then recorded through one segment and the right image perspective through the other segment on the same camera. Because the two perspectives see the object and have the same path length, there is no need to refocus or change the optics between perspectives. The object is seen from both perspectives.

A specific objective of the invention is that the LCD device is sterile for use in surgical procedures and therefore it may be single use, disposable and or withstand autoclaving or other processes that may be used to achieve surgical sterilization.

A specific objective of the invention is that the LCD device should be easy to mount and remove. It may have a snap or adhesive on one side for secure attachment.

A specific objective of the invention is to prevent incorrect orientation of the LCD device. Hence, the LCD device has a notch to advise the surgeon of correct orientation for the placement on the endoscope.

The present invention provides a method of stereoscopic viewing the image displayed on a conventional video monitor using active eyewear. When viewed through LCD shutter glasses, the left side of which is activated at the same time as the left segment of the LCD device and the right side at the same time as the right segment of the LCD device, stereoscopic view is perceived by the viewing person.

A specific objective of the invention is to allow the user, usually a surgeon, wearing the eyewear disclosed in this invention, to be able to see the area around the patient and the operating room clear of the LCD shutter in the glasses. For this objective the LCD shutters are placed only in the upper and inner part of each side of the eyewear, so that the view on the side and when looking down is clear and undistorted. Whereas, when looking at the monitor the view of the left eye is through the left side LCD shutter and that through the right eye is through the right side LCD shutter generating stereoscopic image.

A specific objective of the invention is that the eyewear is sterile. The invention may be single use, disposable or and withstand autoclaving or other processes that may be used to achieve surgical sterilization.

A specific objective of the invention is that the eyewear is adjustable by the surgeon. For this objective the eye wear has a handle on one side that remains sterile and can be used by the surgeon safely without risk of contamination to adjust the eyewear to his or her comfort for accurate viewing of the video monitor.

Other aspects, features and advantages of the invention will become apparent from the following description and from the claims

### BRIEF DESCRIPTION OF SEVERAL VIEWS OF DRAWINGS

In drawings which illustrate embodiments of the present invention,

FIG. 1A shows the conventional endoscope and the camera head

FIG. 1B is a schematic diagram showing a two dimensional single image path system known in the prior art with a endoscope lens system and a camera,

FIG. 2A shows the invention mounted on the endoscope between the endoscope and the camera

FIG. 2B is a schematic diagram showing the single image path system of FIG. 1B with a LCD device according to one embodiment of the present invention placed between the endoscope and the camera to provide stereoscopic viewing,

FIG. 3A is the plan view of the LCD device according to one embodiment of the present invention.

FIG. 3B is another view of the LCD device according to one embodiment of the present invention

FIG. 4 is a schematic view of the eyewear in the present invention to be used to view the video monitor.

FIG. 4B another embodiment of the eyewear used as a ‘clip on’ to surgical eye loops

### DETAILED DESCRIPTION OF THE INVENTION

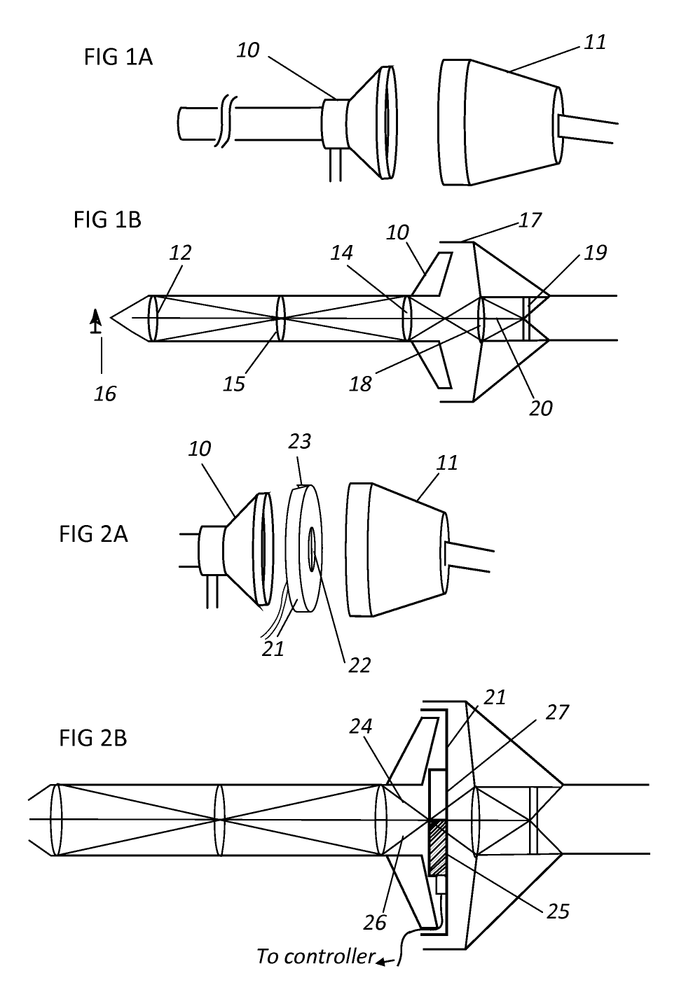

FIG. 1A illustrates a known type of endoscope 10 for two dimensional viewing using a camera 11. In schematic FIG. 1B as shown, the endoscope 10 has entry lenses 12 and exit lenses 14 with other lens 15 there between. Rays from an object 16 pass through the entry pupil and entry lens 12. A coupler 17 couples the endoscope to the camera 11. The rays are focused by a focusing lens 18 on the light sensitive element 19 of the camera 11. The principal ray 20 is shown at the central axis of the endoscope 10.

FIG. 2A shows the endoscope 10 of FIG. 1 modified for stereoscopic viewing system by placing the LCD device 21 with an aperture 22, disclosed herewith, between the endoscope 10 and the camera 11 with notch 23 on LCD device positioned at the top. As in FIG. 2B, the left perspective ray 24 passes through the left segment 25 and the right perspective ray 26 passes through the right segment 27 of the LCD device 21. Thus, the camera sees a different perspective view of the object in the single image path.

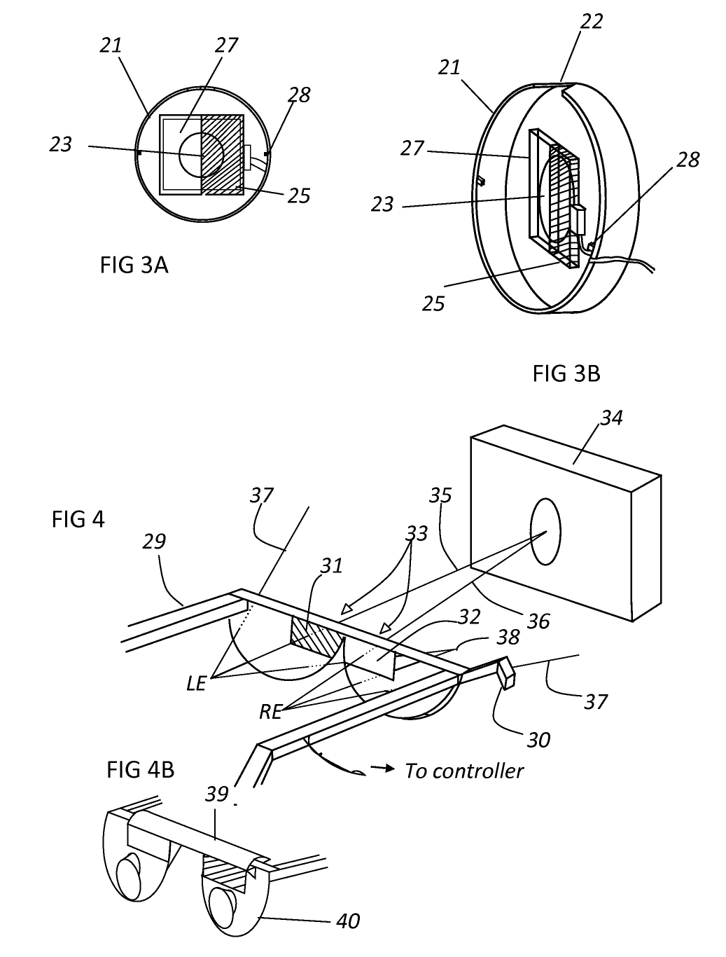

Referring now to FIG. 3, the LCD device 21 is shown with the segments 25 and 27 positioned, a notch 23 is present on the top to provide correct orientation during placement. Two or more snaps 28 help secure the LCD device to the endoscope.

FIG. 4 shows the eyewear 29 disclosed in this invention. The handle 30 is used to mount or un-mount the eyewear sterilely by the surgeon and also make any adjustment during surgery without need of help from the operating room staff. The LCD shutters 31 & 32, similar to the ones used in the LCD device 21 are present in the upper and inner quadrant 33 of the vision field. In this disposition, when the surgeon is looking at the video monitor 34, the principal rays to the left eye (LE) and the right eye (RE), 35 & 36 pass through the respective LCD shutters generating a perception of stereoscopic vision. Vision field rays 37 from the side pass through clear plastic or glass. In this disposition, when the surgeon has to look down at the patient, or has to receive surgical instruments from the nurse, the rays 38 pass through clear plastic or glass providing vision that is unobstructed and clear.

FIG. 4B shows another embodiment of the eyewear 39 that can be clipped on to surgical eye loops 40.

When activated a controller synchronizes activation of the right and left side of the LCD shutters in the eyewear with the right and left segments of the LCD device in the endoscope at a frequency so that there is no observable flicker. Hence, when the right segment of the LCD device is activated and blocks passage of light, the video camera records the left perspective of the object and the video monitor displays the image of left perspective of the object. The controller at the same time activates the right side of the LCD shutter on the eyewear blocking the passage of light, so that the surgeon sees the left perspective of the object from the left side. Likewise, the right perspective of the object is seen by the surgeon when the left segment on the LCD device and left side of the eyewear is activated. The frequency with which the right and left side are toggled can be adjusted on the controller to give the surgeon a view without flicker.

Other changes may be made to the embodiments shown herein without departing from the scope of the present invention which is limited only by the following claims.

We claim,:

1. An LCD device with two segments each segment of which can be activated separately by wired or wireless connection to a controller.

2. The LCD device according to claim 1 that can be attached to a conventional single optic channel endoscope between the endoscope and single lens camera without need for a separate housing.

3. The LCD device according to claim 1, which has a snap or others means such as adhesive to hold it in place.

4. The LCD device according to claim 1, which has a notch or a marker that would provide a guide to appropriate orientation for attachment.

5. The LCD device according to claim 1, which may be sterile, disposable and single use and able to withstand autoclaving or other means of surgical sterilization.

6. An eyewear that has on each side as on each half an LCD shutter synchronized with the LCD device in claim 1 through a wired or wireless connection to a controller.

7. An eyewear according to claim 6, in which the LCD shutter glasses are in the upper inner part of each side of the visual fields.

8. An eyewear according to claim 6, in which the outer and lower part of the visual field has clear glass or plastic.

9. An eyewear according to claim 6, which can be clipped on to surgical eye loops.

10. An eyewear according to claim 6, which has a handle on one or both sides that can be used to mount, un-mount or adjust the eyewear sterilely during surgery

11. An eyewear according to claim 6, that may be sterile, disposable or single use and able to withstand autoclaving or other means of surgical sterilization.
